(19) United States
(12) Patent Application Publication
(10) Pub. No.: US 2025/0302342 A1
(43) Pub. Date: Oct. 2, 2025

(54) CONTINUOUS ANALYTE MONITORING DEVICE

(71) Applicants: Jiangsu Yuwell POCTech Biotechnology Co., Ltd., Zhenjiang (CN); JIANGSU YUEKAI BIOTECHNOLOGY CO., LTD., Nanjing (CN); Zhejiang POCTech Co., Ltd., Huzhou (CN)

(72) Inventors: Liang WANG, Zhenjiang (CN); Yanan ZHANG, Zhenjiang (CN)

(21) Appl. No.: 19/190,834

(22) Filed: Apr. 28, 2025

Related U.S. Application Data
(63) Continuation of application No. PCT/CN2024/122119, filed on Sep. 29, 2024.

(30) Foreign Application Priority Data
Mar. 29, 2024 (CN) 202410377174.3

(51) Int. Cl. A61B 5/145; A61B 5/00; A61B 17/34

(52) U.S. Cl. CPC A61B 5/14503; A61B 5/6849; A61B 17/3468; A61B 5/14532; A61B 2560/063

(57) ABSTRACT

A continuous analyte monitoring device includes an outer housing, a cover, a puncture unit, and a monitoring unit. The outer housing has first and second ends opposite to each other in a first direction. The second end has an implantation opening. The cover is detachably connected to the outer housing. A driving unit is movable along the first direction to perform an implantation operation. The puncture unit is movable along a second direction to perform a needle withdrawal operation. The monitoring unit includes a first monitoring component including a sensor and a second monitoring component including a signal processing module. Prior to use, remove the cover and perform a single trigger to enable the driving unit to move along the first direction, thereby partially inserting the sensor into a host and establishing an electrical connectivity between the first and the second monitoring components.

This application is a continuation of International Patent Application No. PCT/CN2024/122119, filed on Sep. 29, 2024, which claims priority to Chinese patent application No. 202410377174.3, titled “IN VIVO GLUCOSE MONITORING DEVICE” and filed with China National Intellectual Property Administration on Mar. 29, 2024, the entire contents of which are incorporated herein by reference.

### FIELD

The present disclosure belongs to the field of medical device technologies, and more particularly, to a continuous analyte monitoring device.

### BACKGROUND

Continuous Glucose Monitoring (CGM) system is a medical device used to continuously monitor glucose levels of diabetic patients. Compared with conventional glucose monitoring methods, the CGM system provides continuous and detailed data about glucose levels, helping users better manage their glucose states.

When using a CGM product, the users need to place a housing of the CGM product on the skin and then press a trigger button. In this case, a puncture needle and a sensor pin inside the housing move towards and pierce the skin. An electrochemical reaction occurs between biological enzymes on the sensor and an interstitial fluid under the skin, and is converted into an electrical signal. The electrical signal is converted into glucose readings for the users. A sensor for obtaining glucose levels in a host in real time and an electronic component for transmitting signals to a display device are generally integrated within a monitoring unit. After being implanted, the monitoring unit adheres to a surface of the skin of the host to facilitate continuous monitoring.

A structure of the monitoring unit typically includes two types, that is, integrated and split. An integrated monitoring unit is assembled before leaving the factory, with electronic components such as a sensor and a signal transmitter inside the integrated monitoring unit already connected to each other, in such a manner that the users do not need to assemble the integrated monitoring unit and can use the integrated monitoring unit directly. However, production costs of such an integrated monitoring unit are relatively high. In addition, a sterilization process is likely to cause an electronic component failure, which leads to a low yield and unguaranteed reliability.

A split monitoring unit, although offering relatively high reliability, is not fully assembled when the device leaves the factory and needs to be assembled by the users prior to use, which results in a relatively complex structure and cumbersome use steps of the monitoring system and relatively high learning costs of the users, resulting in unsatisfactory use experience.

### SUMMARY

The present disclosure provides a continuous analyte monitoring device to solve technical problems of a complex structure and cumbersome use steps of a monitoring system. The present disclosure adopts the following technical solutions.

A continuous analyte monitoring device includes an outer housing having a first end and a second end that are arranged opposite to each other in a first direction, the second end having an implantation opening; a cover detachably connected to the second end and covering the implantation opening; a driving unit disposed inside the outer housing and movable relative to the outer housing along the first direction to perform an implantation operation; a puncture unit disposed inside the outer housing, the puncture unit being movable along the first direction with the driving unit, and movable relative to the driving unit along a second direction to perform a needle withdrawal operation, the second direction being opposite to the first direction; and a monitoring unit including a first monitoring component and a second monitoring component that are spaced apart from each other inside the outer housing, the first monitoring component including a sensor, and the second monitoring component including a signal processing module. Prior to use, remove the cover and perform a single trigger to enable the driving unit to move along the first direction, thereby partially inserting the sensor into a host and establishing an electrical connectivity between the first monitoring component and the second monitoring component.

Preferably, the first monitoring component further includes a battery configured to be electrically connected to the second monitoring component.

Preferably, the first monitoring component further includes an upper housing, the sensor being disposed at the upper housing, the second monitoring component further includes a lower housing, the signal processing module being disposed at the lower housing, and one of the upper housing and the lower housing is provided with a sealing rib, and the other of the upper housing and the lower housing has a sealing groove in which a seal is disposed, the sealing rib being configured to extend into the sealing groove to abut with the seal for sealing.

Preferably, the outer housing includes a first housing including a connection portion, and a second housing connected to the connection portion. The implantation opening is formed at an end of the second housing away from the first housing. The cover is detachably connected to the second housing to cover the implantation opening. The first monitoring component is fixed inside the first housing. The second monitoring component is fixed inside the second housing.

Preferably, the second housing includes a fixing portion configured for interference fit with the second monitoring component, and the cover is equipped with a support post extending towards the outer housing, the support post being in contact with the second monitoring component to support the second monitoring component.

Preferably, the continuous analyte monitoring device further includes a pushing member configured to drive the driving unit to move along the first direction, and a restoring member configured to drive the puncture unit to move along the second direction, where the pushing member and the restoring member at least partially overlap in the first direction.

Preferably, the continuous analyte monitoring device further includes a trigger unit disposed at the first end, the trigger unit being movable or deformable along the first direction to trigger the driving unit, to enable the first monitoring component or the second monitoring component to move along the first direction.

Preferably, the driving unit includes a first mounting groove internally provided with a restoring member configured to drive the puncture unit to move along the second direction, the puncture unit being disposed in the first mounting groove, and a second mounting groove surrounding an outer periphery of the first mounting groove and internally provided with a pushing member configured to drive the driving unit to move along the first direction.

Preferably, the puncture unit includes a puncture needle, and a needle hub having an engagement groove, the restoring member being disposed in the engagement groove.

Preferably, the needle hub at least partially overlaps the driving unit in the first direction.

Preferably, the continuous analyte monitoring device further includes a support member disposed inside the outer housing and including a first positioning portion and a second positioning portion, the first positioning portion being engaged with the first mounting groove to form a first guiding channel configured to receive the restoring member, and the second positioning portion being engaged with the second mounting groove to form a second guiding channel configured to receive the pushing member.

Preferably, the first positioning portion includes a positioning protrusion protruding towards the engagement groove, and the restoring member has an end connected to the positioning protrusion and another end connected to the puncture unit.

Preferably, the driving unit is provided with a stop portion at an end of the driving unit away from the cover, the stop portion being configured to abut and engage with an end of the puncture unit away from the cover and apply a force in the first direction to the puncture unit.

Preferably, the continuous analyte monitoring device further includes an unlocking structure located in a movement path of the stop portion. When the driving unit moves to an implantation position relative to the unlocking structure along the first direction, the unlocking structure presses the stop portion to disengage the stop portion from the puncture unit.

Preferably, the stop portion includes a fixed end, and a movable end configured to swing around the fixed end, the movable end being provided with a stop segment and a trigger segment, the stop segment extending towards the puncture unit and being configured to abut and engage with the puncture unit, and the trigger segment being configured to engage with the unlocking structure.

Preferably, each of the unlocking structure and/or the stop portion has a guiding transition surface at each of sides of the unlocking structure and/or the stop portion facing each other.

Preferably, the continuous analyte monitoring device further includes a locking member disposed inside the outer housing, the locking member being elastically deformable or movable relative to the outer housing, to have a locked state in which the locking member is engaged with the driving unit to restrict a movement of the driving unit and an unlocked state in which the locking member is disengaged from the driving unit.

Preferably, the outer housing is internally provided with a mounting base, the locking member includes a trigger portion located at a side of the mounting base and an acting portion located at another side of the mounting base and engaged with the driving unit, the continuous analyte monitoring device further includes a trigger unit disposed at the first end and engaged with the trigger portion, and the acting portion is configured to swing around the mounting base to switch into the unlocked state when the trigger portion is pressed by the trigger unit.

By adopting the above technical solutions, the present disclosure provides the following advantageous effects.

In the present disclosure, when the device leaves the factory, the first monitoring component and the second monitoring component of the monitoring unit are separated from each other and are fixed and spaced apart from each other inside the outer housing along the first direction. After the trigger is performed by a user, the first monitoring component can move with the driving unit and the puncture unit along the first direction. An electrical connectivity between the first monitoring component and the second monitoring component is synchronously completed while the puncture unit carries the sensor inside the first monitoring component to be inserted into the host. In this way, assembly of the monitoring unit, the electrical connectivity between the first monitoring component and the second monitoring component, and an implantation of the sensor are simultaneously accomplished through the in single trigger. Therefore, operation steps of the user in an implantation process are simplified, operational difficulty of the product is lowered, and learning costs of the user are lowered. Further, a duration of the entire implantation stage is shortened, which helps to reduce fear of the user while waiting for the implantation. Separately setting the first monitoring component and the second monitoring component allows for individual sterilization treatments. In this way, when the first monitoring component is sterilized, no damage is caused to electronic components of a data processing module in the second monitoring component.

In addition, the cover is fixed at the implantation opening of the outer housing, and the cover is detachably connected to the outer housing. When the device leaves the factory, the cover is fixed at the outer housing to form a relatively sealed environment together with the outer housing to prevent dust and bacteria in an ambient environment from entering the outer housing during warehousing and transportation, ensuring cleanliness of components inside the outer housing. Before the implantation, the user needs to remove the cover from the outer housing to expose the implantation opening, in such a manner that the sensor and the puncture unit inside the outer housing can be inserted into the host through the implantation opening. In this way, to use the continuous analyte monitoring device of the present disclosure, the user only needs to perform a two-step operation. First, the cover needs to be removed from the outer housing to expose the implantation opening, and the implantation opening is tightly attached to the skin. Then, the driving unit is triggered to simultaneously realize the electrical connectivity between the first monitoring component and the second monitoring component and the implantation of the sensor. In this way, the whole implantation process is completed, without performing an additional operation for assembling the upper housing and the lower housing, improving the use experience.

### BRIEF DESCRIPTION OF THE DRAWINGS

The accompanying drawings described here are used to provide a further understanding of the present disclosure and constitute a part of the present disclosure. Exemplary embodiments of the present disclosure and description thereof are used to explain the present disclosure, and do not constitute an improper limitation of the present disclosure.

FIG. 1 is a schematic structural view of a first monitoring component according to an embodiment of the present disclosure.

FIG. 2 is a sectional view of the first monitoring component in FIG. 1.

FIG. 3 is a sectional view of a second monitoring component according to an embodiment of the present disclosure.

FIG. 4 is a schematic structural view of a monitoring unit in a state in which a first monitoring component and a second monitoring component are separated according to an embodiment of the present disclosure.

FIG. 5 is a sectional view of a monitoring unit according to an embodiment of the present disclosure.

FIG. 6 is a sectional view of a partial region of a continuous analyte monitoring device according to an embodiment of the present disclosure.

FIG. 7 is a sectional view of a first housing according to an embodiment of the present disclosure.

FIG. 8 is a sectional view of a partial region of a continuous analyte monitoring device according to an embodiment of the present disclosure.

FIG. 9 is a schematic structural view of a partial region of a continuous analyte monitoring device according to an embodiment of the present disclosure.

FIG. 10 is a sectional view of a second housing and a cover according to an embodiment of the present disclosure.

FIG. 11 is a schematic structural view of the second housing and cover in FIG. 10.

FIG. 12 is a sectional view of a continuous analyte monitoring device in an initial state according to an embodiment of the present disclosure.

FIG. 13 is a sectional view of a continuous analyte monitoring device with a cover removed according to an embodiment of the present disclosure.

FIG. 14 is a sectional view of a first monitoring component and a second monitoring component assembled in an implantation process of a continuous analyte monitoring device according to an embodiment of the present disclosure.

FIG. 15 is a sectional view of a continuous analyte monitoring device after an implantation process is completed according to an embodiment of the present disclosure.

In the accompanying drawings, the reference numbers are listed as follows.
1 outer housing; 11 first housing; 111 engagement protrusion; 112 unlocking structure; 113 trigger unit; 1131 trigger button; 12 second housing; 121 engagement recess; 122 avoidance opening; 123 implantation opening; 124 passage hole; 125 elastic rib; 2 cover; 21 support post; 3 driving unit; 31 first mounting groove; 32 second mounting groove; 33 stop portion; 331 stop segment; 332 trigger segment; 34 snapping hook; 35 fixing opening; 4 puncture unit; 41 needle hub; 411 engagement groove; 42 puncture needle; 5 support member; 51 positioning protrusion; 52 separation rib; 53 accommodation groove; 54 first guiding channel; 55 second guiding channel; 6 monitoring unit; 61 first monitoring component; 611 upper housing; 612 fixing groove; 613 sensor; 6131 substrate; 6132 contact pin; 614 battery; 615 conductive silicone; 616 sealing silicone; 617 sealing rib; 618 snap; 619 through hole; 62 second monitoring component; 621 lower housing; 622 adhesive layer; 623 snap hole; 624 stop rib; 625 signal processing module; 626 sealing groove; 627 seal; 7 pushing member; 8 restoring member; 9 locking member; 91 trigger portion; 92 balance arm; 93 acting portion.

### DETAILED DESCRIPTION

In order to more clearly explain the overall concept of the present disclosure, a detailed description is made below by way of example in conjunction with the accompanying drawings of the specification.

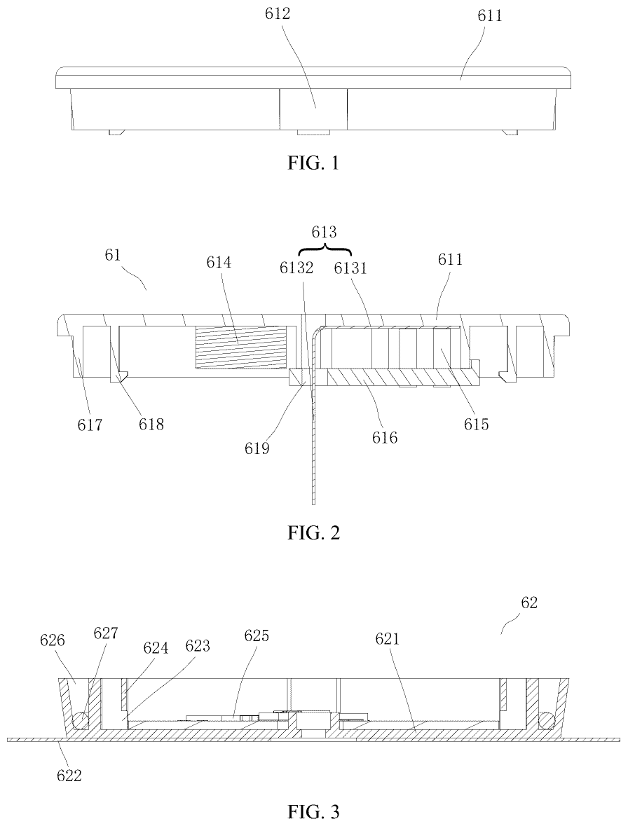

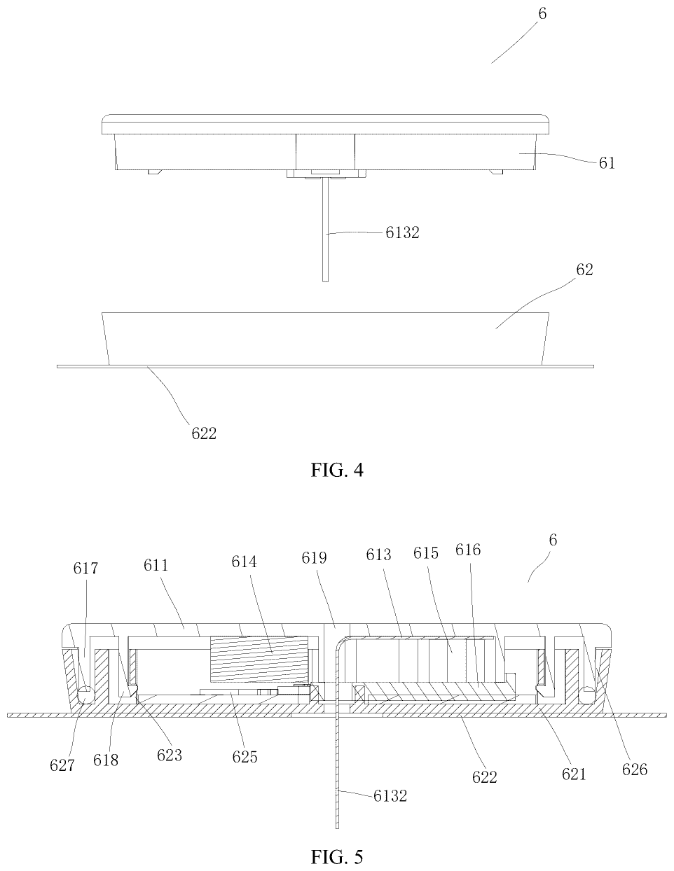

As illustrated in FIG. 1 to FIG. 15, a continuous analyte monitoring device includes an outer housing 1 having a first end and a second end that are arranged opposite to each other in a first direction, the second end having an implantation opening 123; a cover 2 detachably connected to the second end and covering the implantation opening 123; a driving unit 3 disposed inside the outer housing 1 and movable relative to the outer housing 1 along the first direction to perform an implantation operation; a puncture unit 4 disposed inside the outer housing 1, the puncture unit 4 being movable along the first direction with the driving unit 3, and movable relative to the driving unit 3 along a second direction to perform a needle withdrawal operation, the second direction being opposite to the first direction; and a monitoring unit 6 including a first monitoring component 61 and a second monitoring component 62 that are spaced apart from each other inside the outer housing 1. The first monitoring component 61 includes a sensor 613. The second monitoring component 62 includes a signal processing module 625. Prior to use, remove the cover 2 and perform a single trigger to enable the driving unit 3 to move along the first direction, thereby partially inserting the sensor 613 into a host and establishing an electrical connectivity between the first monitoring component 61 and the second monitoring component 62.

It should be understood that, as illustrated in FIG. 1 to FIG. 6, the first direction is parallel to a longitudinal axis of the outer housing 1 and oriented towards the implantation opening 123, while the second direction is parallel to the longitudinal axis of the outer housing 1 and oriented away from the implantation opening 123. That is, the first direction is opposite to the second direction. After a user presses a trigger unit 113, the driving unit 3 carries the puncture unit 4 to move along the first direction together, and the first monitoring component 61 also moves along the first direction under push of the driving unit 3. In this process, the first monitoring component 61 and the second monitoring component 62 are assembled and electrically connected, and the puncture unit 4 carries a contact pin 6132 of the sensor 613 to pierce the skin of the host and enter the host. After the driving unit 3 is moved into place, trigger of the puncture unit 4 is formed, in such a manner that the puncture unit 4 moves along the second direction alone to be withdrawn from the host, completing a needle withdrawal. The second monitoring component 62 is provided with an adhesive layer 622 at a side of the second monitoring component 62 facing towards the implantation opening 123. The second monitoring component 62 adheres and is fixed to a skin surface of the host to be retained at the skin surface of the host, whereas the outer housing 1 and other components inside the outer housing 1 are removed.

It should be noted that a time sequence in which the first monitoring component 61 and the second monitoring component 62 are electrically connected and the sensor 613 is partially inserted into the host is not limited in the present disclosure. For example, during movements of the driving unit 3, the puncture unit 4, and the first monitoring component 61 together along the first direction, the first monitoring component 61 and the second monitoring component 62 may be electrically connected first, at which time the driving unit 3 and the puncture unit 4 have not yet been moved into place. As the movements continue, the puncture unit 4 is inserted into the host to complete the implantation. That is, an electrical connectivity between the first monitoring component 61 and the second monitoring component 62 occurs before the puncture unit 4 is inserted into the host. For example, during the movements of the driving unit 3, the puncture unit 4, and the first monitoring component 61 together along the first direction, the puncture unit 4 is inserted into the host first, at which time the driving unit 3 and the puncture unit 4 have not yet been moved into place. As the movements continue, an upper housing 611 and a lower housing 621 are assembled, meanwhile, the electrical connectivity between the first monitoring component 61 and the second monitoring component 62 is completed. That is, the insertion of the puncture unit 4 into the host occurs before the electrical connectivity between the first monitoring component 61 and the second monitoring component 62 occurs. In addition, the above two steps may also occur simultaneously.

However, regardless of the situations in any of the above examples, the user only needs to perform the single trigger, without carrying out any additional operations. Consequently, the situations described in the above examples fall within the scope of protection of the present disclosure.

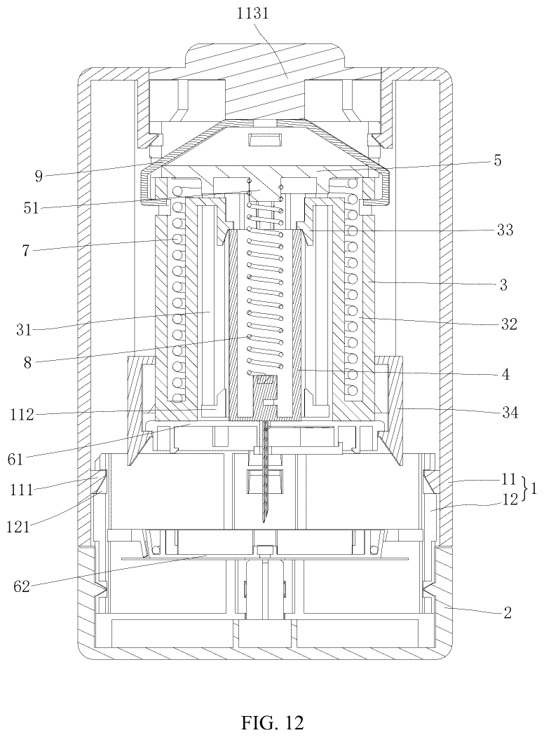

As illustrated in FIG. 12, the first monitoring component 61 and the second monitoring component 62 of the monitoring unit 6 are separated from each other when the device leaves the factory, and are fixed inside the outer housing 1 separately. After the trigger is performed by the user, the upper housing 611 can move with the driving unit 3 and the puncture unit 4 along the first direction. An operation of the puncture unit 4 carrying the sensor 613 inside the first monitoring component 61 to be inserted into the host and the electrical connectivity between the first monitoring component 61 and the second monitoring component 62 are synchronously completed. In this way, the electrical connectivity between the first monitoring component 61 and the second monitoring component 62 and an implantation of the sensor 613 are simultaneously accomplished through the single trigger. Therefore, operation steps of the user in the implantation process are simplified, operational difficulty of the product is lowered, and learning costs of the user are lowered. Further, a duration of the entire implantation stage is shortened, which helps to reduce fear of the user while waiting for the implantation.

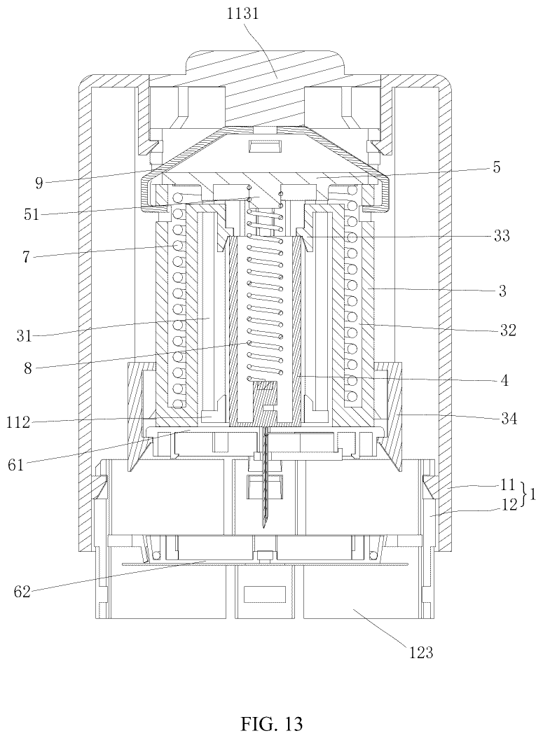

In addition, as illustrated in FIG. 12 and FIG. 13, the cover 2 is fixed at the implantation opening 123 of the outer housing 1, the cover 2 is detachably connected to the outer housing 1. When the device leaves the factory, the cover 2 is fixed at the outer housing 1 to form a relatively sealed environment together with the outer housing 1 to prevent dust and bacteria in an ambient environment from entering the outer housing 1 during warehousing and transportation, ensuring cleanliness of components inside the outer housing 1. Before the implantation, the user needs to remove the cover 2 from the outer housing 1 to expose the implantation opening 123, in such a manner that the sensor 613 and the puncture unit 4 inside the outer housing 1 can be inserted into the host through the implantation opening 123.

In this way, to use the continuous analyte monitoring device of the present disclosure, the user only needs to perform a two-step operation. First, the cover 2 needs to be removed from the outer housing 1 to expose the implantation opening 123, and the implantation opening 123 is tightly attached to the skin. Then, the driving unit 3 is triggered to simultaneously realize the electrical connectivity between the first monitoring component 61 and the second monitoring component 62 and the implantation of the sensor 613. In this way, the whole implantation process is completed, without performing an additional operation for assembling the upper housing and the lower housing 621, improving the use experience.

As a preferred embodiment of the present disclosure, as illustrated in FIG. 2, the first monitoring component 61 further includes a battery 614 configured to be electrically connected to the second monitoring component 62. The battery 614 mainly serves to supply power to the signal processing module 625. Before the user triggers the implantation, the battery 614 is not electrically connected to the signal processing module 625 since the first monitoring component 61 and the second monitoring component 62 are separated from each other. The battery 614 is electrically connected to the signal processing module 625 only after the user triggers the implantation. In this way, an energy loss of the battery 614 before the device is used by the user can be reduced to extend a warehousing duration.

Preferably, as illustrated in FIG. 2, FIG. 3, and FIG. 5, the first monitoring component 61 includes the upper housing 611. The sensor 613 is disposed at the upper housing 611. The second monitoring component 62 includes the lower housing 621. The signal processing module 625 is disposed at the lower housing 621. The upper housing 611 is engaged with and fixed to the lower housing 621 to reduce difficulty of fixing the upper housing 611 and the lower housing 621, which eliminates a need to connect the upper housing 611 and the lower housing 621 manually. Fixation can be accomplished by relying on a force from the driving unit 3 only. Specifically, as illustrated in FIG. 2 and FIG. 3, the upper housing 611 is provided with a snap 618, and the lower housing 621 has a snap hole 623. When moving with the driving unit 3 along the first direction, the upper housing 611 collides with the lower housing 621. Under an impact force, the snap 618 slides into the snap hole 623, in such a manner that the upper housing 611 is engaged with and fixed to the lower housing 621. Of course, the snap 618 may also be disposed at the lower housing 621, and correspondingly, the snap hole 623 may be formed at the upper housing 611, which likewise enables the upper housing 611 to engage with and fix to the lower housing 621. Specifically, as illustrated in FIG. 3, the snap 618 has a guiding surface at a side of the snap 618 facing towards the snap hole 623, and a stop rib 624 is disposed at a side of the snap hole 623 close to the snap 618. The snap 618 comes into contact with the stop rib 624, and then deforms and expands outwards under pressing of the stop rib 624. As the snap 618 continues to move to the snap hole 623, the pressing of the stop rib 624 disappears, and thus the snap 618 experiences a deformation recovery due to its own elasticity and is engaged in the snap hole 623.

The upper housing 611 has a through hole 619 formed corresponding to the contact pin 6132 of the sensor 613. During a movement of the puncture unit 4 along the first direction, a puncture needle 42 of the puncture unit 4 passes through the through hole 619 of the upper housing 611 and surrounds the contact pin 6132 of the sensor 613. The upper housing 611 and the lower housing 621 are assembled during a movement of the driving unit 3. In addition, the puncture unit 4 carries the contact pin 6132 of the sensor 613 to pierce the skin of the host and enter the host. After the driving unit 3 is moved into place, the trigger of the puncture unit 4 is formed, in such a manner that the puncture unit 4 moves along the second direction alone to be withdrawn from the host, completing the needle withdrawal. The lower housing 621 is provided with the adhesive layer 622 at a side of the lower housing 621 facing towards the implantation opening 123. The lower housing 621 adheres and is fixed to the skin surface of the host to be retained at the skin surface of the host, whereas the outer housing 1 and other components inside the outer housing 1 are removed.

Preferably, as illustrated in FIG. 2 and FIG. 3, one of the upper housing 611 and the lower housing 621 is provided with a sealing rib 617, and the other of the upper housing 611 and the lower housing 621 has a sealing groove 626. A seal 627 is disposed in the sealing groove 626. The sealing rib 617 is configured to extend into the sealing groove 626 to abut with the seal 627 for sealing. The sealing rib 617 is arranged corresponding to the seal 627 in the sealing groove 626. When the upper housing 611 is in contact with the lower housing 621 in the first direction, the sealing rib 617 is inserted into the sealing groove 626 to abut with the seal 627 for pressing and deforming the seal 627, realizing the sealing between the upper housing and the lower housing. Specifically, as illustrated in FIG. 2 and FIG. 3, the sealing rib 617 is disposed at the upper housing 611, the sealing groove 626 is formed at the lower housing 621, and the seal 627 is an O-type sealing ring disposed in the sealing groove 626. Alternatively, positions of the sealing rib 617 and the sealing groove 626 may be interchanged. That is, the sealing rib 617 is disposed at the lower housing 621, while the sealing groove 626 is correspondingly formed at the upper housing 611. The present disclosure is not limited in this regard. Preferably, as illustrated in FIG. 3, groove walls of the sealing groove 626 at two sides of the sealing groove 626 extend obliquely to enable a width of an opening of the sealing groove 626 to be greater than a width of a bottom of the sealing groove 626, providing guidance for the insertion of the sealing rib 617. Consequently, the sealing rib 617 can be inserted into and engaged with the sealing groove 626 more smoothly to reduce a sense of jamming generated during the insertion and the engagement.

Further, as illustrated in FIG. 2, the sensor 613 includes a sheet-like substrate 6131 and the contact pin 6132 extending downwards and integrating a plurality of electrodes. The contact pin 6132 is configured to be inserted into human epidermis to analyze concentration indexes of glucose and other analytes of an individual through an electrochemical reaction in an interstitial fluid inside the epidermis. The substrate 6131 is provided with conductive silicone 615 at a lower side of the substrate 6131. The conductive silicone 615 is configured to transmit an electrical signal of an electrochemical reaction of the sensor 613 to the signal processing module 625. The conductive silicone 615 is further provided with sealing silicone 616 at a lower side of the conductive silicone 615. The sealing silicone 616 is mainly used for waterproof and sealing of an electrode contact of the sensor 613, and cooperates with the conductive silicone 615 to realize an electrical connectivity to the signal processing module 625 while achieving sealing.

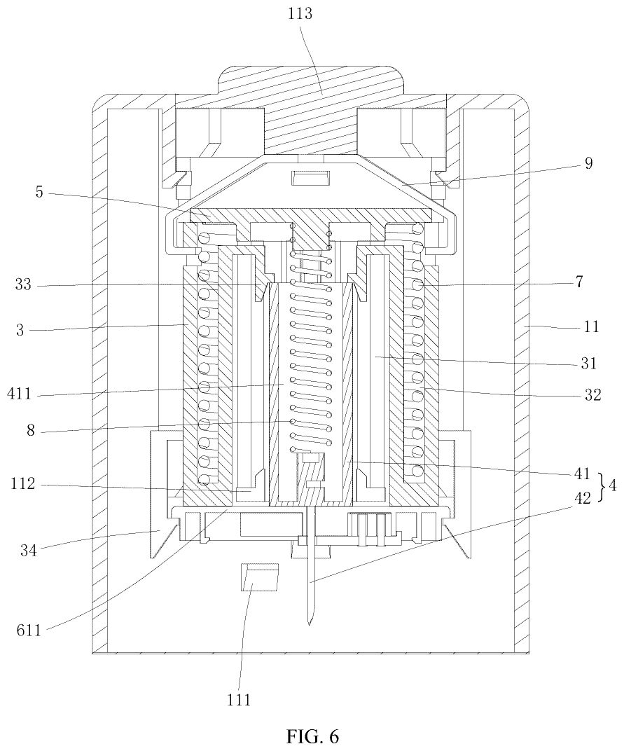

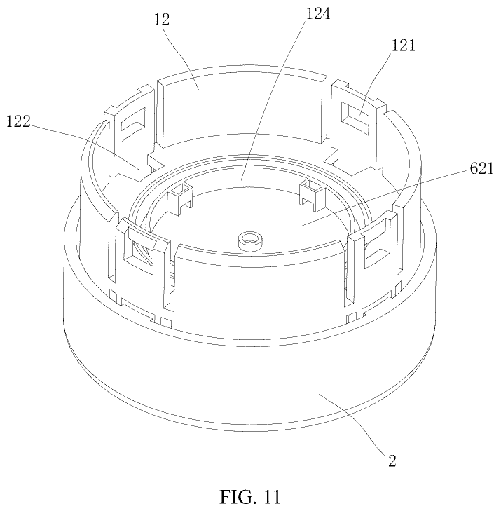

As a preferred embodiment of the present disclosure, as illustrated in FIG. 6, FIG. 10, and FIG. 12, the outer housing 1 includes a first housing 11 and a second housing 12. The first housing 11 includes a connection portion. The second housing 12 is connected to the connection portion. The implantation opening 123 is formed at an end of the second housing 12 away from the first housing 11. The cover 2 is detachably connected to the second housing 12 to cover the implantation opening 123. The first monitoring component 61 is fixed inside the first housing 11. The second monitoring component 62 is fixed inside the second housing 12. Specifically, as illustrated in FIG. 10, FIG. 11, and FIG. 12, the cover 2 and the second housing 12 are fixed through a snap connection. Of course, the cover 2 may also be fixed to the second housing 12 through a threaded connection, interference fit, a pluggable connection, and the like. The present disclosure is not limited to any of these examples.

The outer housing 1 has a split structure. The driving unit 3, the puncture unit 4, and the first monitoring component 61 are fixed within the first housing 11. The second monitoring component 62 is fixed within the second housing 12. In this way, the continuous analyte monitoring device is divided into two modules. Components having relatively high sterilization requirements, such as the puncture unit 4 and the sensor 613, are concentrated in one module (within the first housing 11). Therefore, before leaving the factory, two modules, the first housing 11 and the second housing 12, can be separately sterilized using different processes or at different levels. After the sterilization, the first housing 11 and the second housing 12 are assembled into an entirety. This prevents an issue of a failure of the sensor 613 or an electronic component during the sterilization. Moreover, the second housing 12 and the first housing 11 have been fixed when the device leaves the factory. Consequently, instead of performing any fixing operations on the second housing 12 and the first housing 11 prior to use, the user only needs to directly perform the trigger, which reduces operation steps, improving the use experience.

As illustrated in FIG. 6 and FIG. 12, in an embodiment, the second housing 12 has an engagement recess 121 at a side wall of the second housing 12, and the first housing 11 is provided with an engagement protrusion 111 at a side wall of the first housing 11, allowing the second housing 12 to engage with and fix to the first housing 11. The engagement protrusion 111 may be disposed at an inner wall of the first housing 11, and thus the side wall of the second housing 12 can be placed into the first housing 11 to be fixed to the first housing 11. The engagement protrusion 111 may also be disposed at an outer wall of the first housing 11, and thus the first housing 11 can be placed into the second housing 12 to be fixed to the second housing 12. The first housing 11 and the second housing 12 may also be fixed by other means, e.g., a threaded connection. The present disclosure is not limited in this regard.

Further, as illustrated in FIG. 10 and FIG. 11, the second housing 12 includes a fixing portion configured for interference fit with the second monitoring component 62. Specifically, as illustrated in FIG. 10 and FIG. 11, the second housing 12 has a passage hole 124. A plurality of elastic ribs 125 are arranged at intervals at an edge of the passage hole 124. A mounting site is formed by the plurality of elastic ribs 125 and configured to fix the lower housing 621 of the second monitoring component 62. After the lower housing 621 is mounted, the elastic rib 125 is pressed outwards, in which case the elastic rib 125 presses inwards under its own elastic force to clamp and fix the lower housing 621. When the lower housing 621 is pushed by the upper housing 611 in the first direction or when the adhesive layer 622 of the lower housing 621 adheres to the skin of the host, the lower housing 621 can be free from clamping of the elastic rib 125 to fall off from the second housing 12.

As illustrated in FIG. 1 and FIG. 6, the driving unit 3 is provided with a snapping hook 34, and the upper housing 611 has a fixing groove 612 at a peripheral side of the upper housing 611. The snapping hook 34 is engaged with and fixed to the fixing groove 612 to enable the upper housing 611 to be fixed to and move with the driving unit 3. Groove walls of the fixing groove 612 at two sides of the fixing groove 612 can stop and restrict a position of the snapping hook 34 to restrict the upper housing 611 from rotating relative to the driving unit 3. Further, the second housing 12 has an avoidance opening 122 at an outer side of the passage hole 124. A trigger rib is disposed at a side of the avoidance opening 122 close to a center of the second housing 12. When the driving unit 3 carries the upper housing 611 to move to the second housing 12, the trigger rib presses the snapping hook 34, causing the snapping hook 34 to expand and deform outwards. In this way, the snapping hook 34 is detached from the fixing groove 612 to release the upper housing 611 and the snapping hook 34 enters the avoidance opening 122.

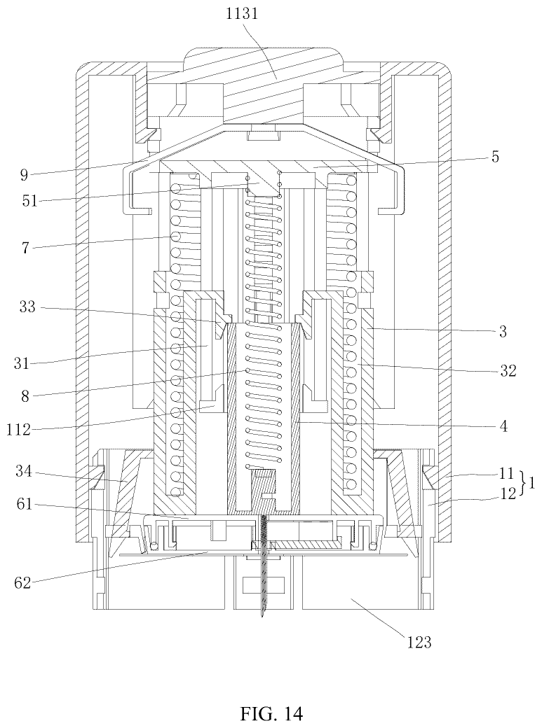

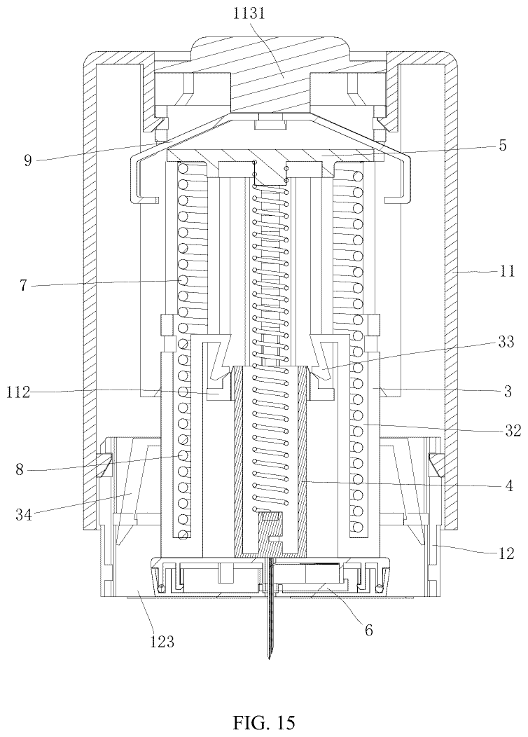

Preferably, as illustrated in FIG. 12, the cover 2 is equipped with a support post 21 extending towards the outer housing 1, the support post 21 is in contact with the second monitoring component 62 to support the second monitoring component 62. As illustrated in FIG. 12, the support post 21 provides support for the second monitoring component 62 before the user removes the cover 2, ensuring that the second monitoring component 62 is stably fixed in the second housing 12 to avoid a detachment of the second monitoring component 62. Specifically, as illustrated in FIG. 13, the user removes the cover 2 prior to use. As illustrated in FIG. 14, after the user presses the trigger unit 113, the driving unit 3 is free from a movement restriction and carries the upper housing 611 to move along the first direction. During the movement, as illustrated in FIG. 14, the upper housing 611 is in contact with the lower housing 621 and fixation and sealing are realized, while the snapping hook 34 is pressed by the trigger rib at the second housing 12 to release the first monitoring component 61. The second monitoring component 62 is pushed by the first monitoring component 61 to break away from the clamping of the elastic rib 125. In this case, the upper housing 611 and the lower housing 621 are assembled, and the first monitoring component 61 and the second monitoring component 62 are electrically connected. As illustrated in FIG. 15, the driving unit 3 carries the entire monitoring unit 6 to continue to move along the first direction, until the puncture unit 4 pierces into the host, and the adhesive layer 622 is in contact with and adheres to the skin surface of the host. Preferably, two or a plurality of snaps 618, engagement protrusions 111, and snapping hooks 34 may be provided as desired and arranged at intervals circumferentially to form a uniform fixation force in a circumferential direction of the second housing 12 and a circumferential direction of the monitoring unit 6 to avoid tilting.

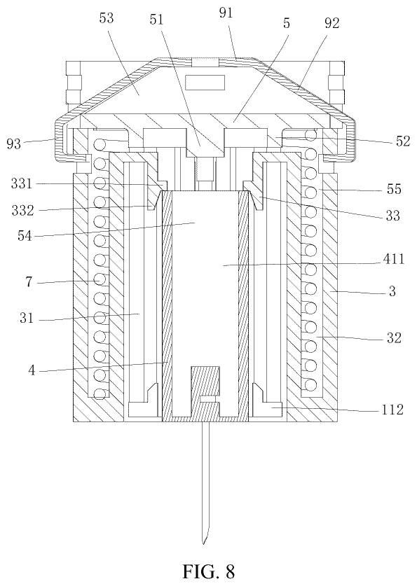

In a preferred embodiment, as illustrated in FIG. 6 and FIG. 8, the continuous analyte monitoring device includes a pushing member 7 configured to drive the driving unit 3 to move along the first direction and a restoring member 8 configured to drive the puncture unit 4 to move along the second direction. The pushing member 7 at least partially overlaps the restoring member 8 in the first direction. In an axial direction of the outer housing 1, the restoring member 8 at least partially overlaps the pushing member 7, which can improve compactness of a structural arrangement, and help to reduce an overall axial dimension of the continuous analyte monitoring device, realizing miniaturization. Specifically, as illustrated in FIG. 6, each of the pushing member 7 and the restoring member 8 is a spring. In an initial state, the pushing member 7 is in a pressed state, forming a pushing force on the driving unit 3 in the first direction. The restoring member 8 is not deformed in the initial state. During a movement of the driving unit 3 and the puncture unit 4 along the first direction, the restoring member 8 is gradually elongated to form a pulling force on the puncture unit 4 in the second direction. In this way, the puncture unit 4 is ensured not to perform the needle withdrawal before an end of the implantation process to avoid an implantation failure, improving an implantation success rate. Further, fatigue of the restoring member 8 caused by the restoring member 8 being in a deformed state for a long period of time can be avoided, improving a needle withdrawal effect. Alternatively, the restoring member 8 may be designed to be elongated in the initial state to enhance a driving force of the restoring member 8 on the puncture unit 4.

Preferably, as illustrated in FIG. 6, the continuous analyte monitoring device further includes the trigger unit 113 disposed at an end of the outer housing 1 away from the implantation opening 123. The trigger unit 113 is movable or deformable along the first direction to trigger the driving unit 3. Each of the first monitoring component 61 and the second monitoring component 62 is movable along the first direction. The user presses the trigger unit 113 in the first direction, in such a manner that the trigger unit 113 forms trigger for the driving unit 3. After being triggered, the driving unit 3 also carries the puncture unit 4 to move towards the implantation opening 123 along the first direction and pushes the first monitoring component 61 and the second monitoring component 62 to move to the implantation opening 123 along the first direction to come into contact with the skin of the patient for completing adhesion. In this embodiment, a direction of pressing the trigger unit 113, an implantation direction of the puncture unit 4, and movement directions of the first monitoring component 61 and the second monitoring component 62 are identical, which not only makes an arrangement of a needle assist structure inside the outer housing 1 simpler and more compact, but also makes a movement of each component more reliable and helps to ensure stability of the movement of the puncture unit 4, preventing the pain felt by the user from being increased due to shaking during the implantation of the puncture unit 4.

As a preferred embodiment of the present disclosure, as illustrated in FIG. 6, the driving unit 3 has a first mounting groove 31 and a second mounting groove 32 surrounding an outer periphery of the first mounting groove 31. The puncture unit 4 is disposed in the first mounting groove 31. The first mounting groove 31 is internally provided with a restoring member 8 configured to drive the puncture unit 4 to move along the second direction. The second mounting groove 32 is internally provided with a pushing member 7 configured to drive the driving unit 3 to move along the first direction. The puncture unit 4 is disposed in the first mounting groove 31.

The driving unit 3 has a space for accommodating the pushing member 7 and the restoring member 8, allowing an arrangement of the pushing member 7 and the restoring member 8 to be more centralized. In addition, the pushing member 7 surrounds an outer periphery of the restoring member 8. Compared with arranging the pushing member 7 and the restoring member 8 in parallel, arranging the pushing member 7 to surround the outer periphery of the restoring member 8 can reduce a space occupied in a radial direction of the outer housing 1, which helps to reduce a radial dimension of the outer housing 1, realizing the miniaturization. In addition, arranging the second mounting groove 32 to surround the first mounting groove 31 also realizes that, after the restoring member 8 and the pushing member 7 are mounted, the restoring member 8 and the pushing member 7 partially overlap each other in the first direction (the axial direction of the outer housing 1), which helps to reduce a space occupied in the axial direction. Reducing the dimension of the outer housing 1 in both the axial direction and the radial direction makes the entire continuous analyte monitoring device more compact and lightweight, enhancing convenience for use and storage. Further, groove walls of the first mounting groove 31 and the second mounting groove 32 can also provide guidance for the restoring member 8 and the pushing member 7, which can reduce shaking of the puncture unit 4 during the needle insertion or the needle withdrawal and keep the puncture unit 4 moving along an axis, reducing the pain felt by the user during the needle insertion and the needle withdrawal. Specifically, as illustrated in FIG. 6, the driving unit 3 has a cylindrical structure. The driving unit 3 has an inner wall and an outer wall. The first mounting groove 31 is formed by the inner wall in a center region of the driving unit 3. The second mounting groove 32 is formed between the inner wall and the outer wall.

Further, as illustrated in FIG. 6, the puncture unit 4 includes a puncture needle 42 and a needle hub 41. The needle hub 41 has an engagement groove 411. The restoring member 8 is disposed in the engagement groove 411. The needle hub 41 has the engagement groove 411. The engagement groove 411 has an engagement opening away from the implantation opening 123. The engagement opening is configured for placement of the restoring member 8. Formation of the engagement groove 411 further reduces a space for the placement of the restoring member 8, which improves a position restriction on and guidance for the restoring member 8, making a deformation of the restoring member 8 more reliable. Specifically, as illustrated in FIG. 6, the engagement groove 411 has a depth greater than ½ of a depth of the restoring member 8 to further improve the position restriction on and the guidance for the restoring member 8.

Preferably, as illustrated in FIG. 6, the needle hub 41 at least partially overlaps the driving unit 3 in the first direction. The needle hub 41 and the driving unit 3 are coaxially arranged and partially overlap each other in the axial direction, which can further improve compactness of the structure and optimize a layout of the structure, realizing a miniaturized design.

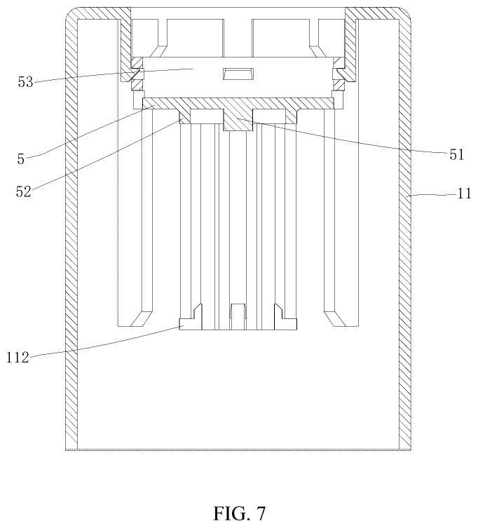

In a preferred embodiment, as illustrated in FIG. 6, FIG. 7, and FIG. 8, the continuous analyte monitoring device includes a support member 5 disposed inside the outer housing 1. The support member 5 includes a first positioning portion and a second positioning portion. The first positioning portion is engaged with the first mounting groove 31 to form a first guiding channel 54 configured to receive the restoring member 8. The second positioning portion is engaged with the second mounting groove 32 to form a second guiding channel 55 configured to receive the pushing member 7. The support member 5 is engaged with the driving unit 3 to form the first guiding channel 54 and the second guiding channel 55 that are configured to receive the restoring member 8 and the pushing member 7, respectively. The first guiding channel 54 and the second guiding channel 55 are coaxial and arranged around each other, in such a manner that the first guiding channel 54 and the second guiding channel 55 overlap over a larger region in height, which makes an internal structure more compact, reducing a dimension of the product, and improving guidance for the restoring member 8 and the pushing member 7 to ensure that the puncture unit 4 moves axially without tilting. The puncture unit 4 is disposed in the first guiding channel 54.

Preferably, as illustrated in FIG. 7, the support member 5 is provided with a separation rib 52 between the first guiding channel 54 and the second guiding channel 55 to separate the first guiding channel 54 from the second guiding channel 55, preventing the restoring member 8 and the pushing member 7 from interfering with each other during deformation. Further, as illustrated in FIG. 7 and FIG. 8, the first positioning portion includes a positioning protrusion 51 protruding towards the engagement groove 411, and the restoring member 8 has an end connected to the positioning protrusion 51 and another end connected to the puncture unit 4. The positioning protrusion 51 can provide positioning for mounting of the restoring member 8 to reduce difficulty of mounting the restoring member 8, and provide guidance during the deformation of the restoring member 8 to prevent the restoring member 8 from tilting and being deformed. Specifically, as illustrated in FIG. 6, the restoring member 8 is a spring, and has an end arranged around an outer periphery of the positioning protrusion 51 and another end abutting with or fixedly connected to the puncture unit 4. Preferably, as illustrated in FIG. 6 and FIG. 8, the support member 5 and the driving unit 3 are coaxially arranged.

In a preferred embodiment of the present disclosure, as illustrated in FIG. 6 and FIG. 8, the driving unit 3 is provided with a stop portion 33 at an end of the driving unit 3 away from the cover 2. The stop portion 33 is configured to abut and engage with an end of the puncture unit 4 away from the cover 2 to apply a force in the first direction to the puncture unit 4. On the one hand, the stop portion 33 can abut and engage with the end of the puncture unit 4 away from the cover 2 to stop the puncture unit 4, restricting the puncture unit 4 from moving along the second direction. Therefore, positional stability of the puncture unit 4 before the implantation is ensured to prevent the puncture unit 4 from performing the needle withdrawal in advance. On the other hand, after being triggered, the driving unit 3 moves along the first direction and transmits power to the puncture unit 4 through the stop portion 33 to drive the puncture unit 4 to move along the first direction for completing the needle insertion. In this way, an integration of functions of position restriction and driving of the stop portion 33 is realized. Such a multi-purpose design simplifies the structure, saves a space inside the outer housing 1, and lowers costs. Specifically, as illustrated in FIG. 8, the stop portion 33 is located in the first mounting groove 31 and configured to abut with the puncture unit 4.

Preferably, as illustrated in FIG. 6, FIG. 8, and FIG. 12 to FIG. 15, the continuous analyte monitoring device further includes an unlocking structure 112 located in a movement path of the stop portion 33. When the driving unit 3 moves to an implantation position relative to the unlocking structure 112 along the first direction, the unlocking structure 112 presses the stop portion 33 to disengage the stop portion 33 from the puncture unit 4. The unlocking structure 112 is located in the movement path of the stop portion 33 in the first direction. Further, the unlocking structure 112 is fixed in position and does not move with the driving unit 3. Therefore, when the driving unit 3 carries the puncture unit 4 to move into place along the first direction, the unlocking structure 112 can press the stop portion 33, in such a manner that the stop portion 33 releases a restriction on the puncture unit 4, automatically triggering unlocking of the puncture unit 4. As a result, the puncture unit 4 performs the needle withdrawal in the second direction, which eliminates a need for the user to additionally perform the needle withdrawal operation. The single trigger automatically completes consecutively performing the implantation and the needle withdrawal, which simplifies the operation steps of the user and reduces the operational difficulty, greatly improving the use experience. Specifically, as illustrated in FIG. 8, the stop portion 33 includes a fixed end and a movable end configured to swing around the fixed end. The movable end is provided with a stop segment 331 and a trigger segment 332. The stop segment 331 extends towards the puncture unit 4 and is configured to abut and engage with the puncture unit 4. The trigger segment 332 is configured to engage with the unlocking structure 112.

The fixed end of the stop portion 33 is fixed to a body of the driving unit 3 or the outer housing 1, while the movable end of the stop portion 33 is free, which provides a strong elastic deformation ability for the stop portion 33. When pressed, the movable end can swing around the fixed end. The movable end is provided with the stop segment 331 and the trigger segment 332. When the stop portion 33 moves into place in the first direction, the trigger segment 332 stops with the unlocking structure 112 and is pressed, causing the movable end to be displaced, and the stop segment 331 moves with the movable end to be disengaged from the puncture unit 4, releasing a stop and a position restriction on the puncture unit 4. In this case, the puncture unit 4 can be driven by the restoring member 8 to move along the second direction to realize the needle withdrawal.

Further, as illustrated in FIG. 8, each of the unlocking structure 112 and/or the stop portion 33 has a guiding transition surface at each of sides of the unlocking structure 112 and/or the stop portion 33 facing each other. With the guiding transition surface, an engagement between the unlocking structure 112 and the stop portion 33 becomes smoother, which reduces the sense of jamming generated after the unlocking structure 112 comes into contact with the stop portion 33, and the stop portion 33 is able to expand and deform outwards more smoothly, which helps to improve timeliness of triggering a needle withdrawal movement and shortens a duration of the entire implantation process. In addition, a relative positional deviation between the stop portion 33 and the unlocking structure 112 caused by manufacturing and assembly errors can also be reduced to enable the stop portion 33 and the unlocking structure 112 to be reliably engaged with each other. Preferably, the guiding transition surface may be an inclined surface, an arc surface, or other irregular curved surfaces, as long as guidance can be provided for the engagement between the stop portion 33 and the unlocking structure 112. The present disclosure is not limited in this regard.

In the above embodiment, the stop portion 33 is elastically deformable to be disengaged from the puncture unit 4. In another embodiment, the stop portion 33 may also be provided as a structure movable relative to the puncture unit 4, in which case when triggered by the unlocking structure 112, the triggering structure pushes the stop portion 33 to move to disengage from the puncture unit 4. The present disclosure is not limited in this regard.

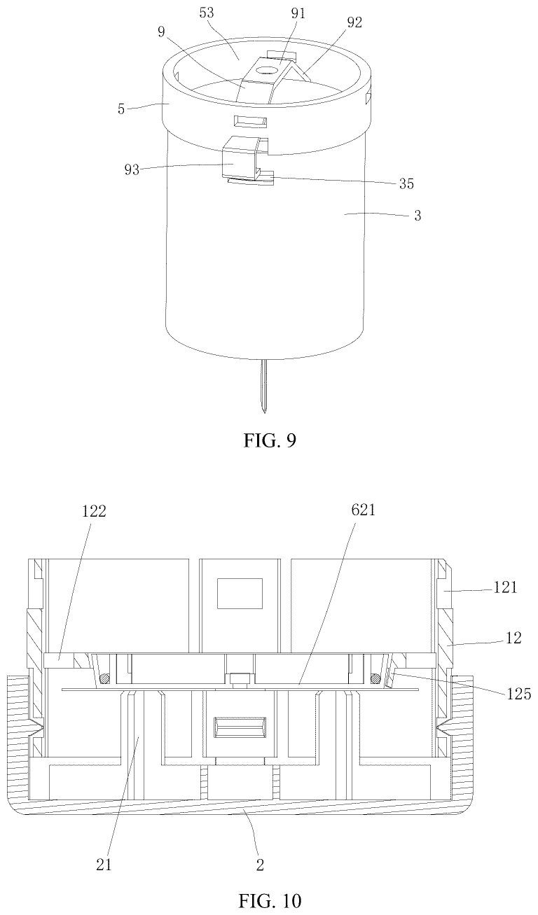

In a preferred embodiment, as illustrated in FIG. 6, FIG. 8, and FIG. 9, the continuous analyte monitoring device further includes a locking member 9 disposed inside the outer housing 1. The locking member 9 is elastically deformable or movable relative to the outer housing 1, to have a locked state in which the locking member 9 is engaged with the driving unit 3 to restrict a movement of the driving unit 3 and an unlocked state in which the locking member 9 is disengaged from the driving unit 3. The locking member 9 is configured to lock the driving unit 3 inside the outer housing 1 before the trigger unit 113 is triggered by the user, to prevent the driving unit 3 from moving. Therefore, overall reliability of the use of the continuous analyte monitoring device is improved to avoid scrapping of the product occurred prior to use due to accidental triggering. Only after the user triggers the trigger unit 113, the locking member 9 unlocks the driving unit 3. Specifically, as illustrated in FIG. 6 and FIG. 8, the trigger unit 113 is a trigger button 1131 disposed at the end of the outer housing 1 away from the implantation opening 123, and the locking member 9 is arranged between the trigger button 1131 and the driving unit 3. In the initial state, the locking member 9 is engaged with the driving unit 3 to lock a position of the driving unit 3. When the user presses the trigger button 1131, as illustrated in FIG. 14, the trigger button 1131 can push the locking member 9 to move or undergo an elastic deformation to disengage from the driving unit 3. As illustrated in FIG. 12, the trigger button 1131 includes a pressing protrusion protruding towards the locking member 9.

Further, as illustrated in FIG. 8 and FIG. 9, the outer housing 1 is internally provided with a mounting base. The locking member 9 includes a trigger portion 91 located at a side of the mounting base and an acting portion 93 located at another side of the mounting base. The acting portion 93 is engaged with the driving unit 3. The continuous analyte monitoring device further includes the trigger unit 113 disposed at the end of the outer housing 1 away from the implantation opening 123 and engaged with the trigger portion 91. When the trigger unit 113 presses the trigger portion 91, the acting portion 93 may swing around the mounting base to switch into the unlocked state. The trigger portion 91 and the acting portion 93 of the locking member 9 are located at two sides of the mounting base, forming a lever structure through an engagement with the mounting base. When the trigger portion 91 located at the side of the mounting base is pressed by the trigger unit 113, the acting portion 93 at the other side of the mounting base may swing around the mounting base. Specifically, as illustrated in FIG. 8 and FIG. 9, a center region of the locking member 9 has a contact plane configured to be in contact with the trigger button 1131 to make contact between the locking member 9 and the trigger button 1131 more stable. The contact plane is provided with a balance arm 92 at a peripheral side of the contact plane, the balance arm 92 extends obliquely towards the implantation opening 123. The balance arm 92 extends from the side of the mounting base to the other side of the mounting base. The contact plane is the trigger portion 91. An end away from the contact plane is the acting portion 91. Preferably, the locking member 9 is coaxially arranged with the driving unit 3 and the trigger unit 113. The acting portion 93 is provided with a locking snapping hook 34 extending radially inwards along the driving unit 3. The driving unit 3 has a fixing opening 35 at a side wall of the driving unit 3. The locking snapping hook 34 extends into the fixing opening 35 to realize a hook engagement between the locking snapping hook 34 and the fixing opening 35. As an example, in FIG. 8, when the contact plane is pressurized to move downwards, the acting portion 93 at a lower end of the balance arm 92 expands outwards around the mounting base, in such a manner that the fixing snapping hook 34 falls out of the fixing opening 35, completing unlocking of the driving unit 3.

Preferably, the continuous analyte monitoring device includes the support member 5. At least part of the support member 5 forms the mounting base. Specifically, as illustrated in FIG. 8 and FIG. 9, the support member 5 has an accommodation groove 53. A bottom wall of the accommodation groove 53 is engaged with the driving unit 3 to form the second guiding channel 55 configured to receive the pushing member 7 and the first guiding channel 54 configured to receive the restoring member 8. The contact plane is located in the accommodation groove 53, the accommodation groove 53 provides a space for upward and downward movements of the contact plane. A side wall of the accommodation groove 53 has a passage opening for the balance arm 92 to pass through. The bottom wall of the accommodation groove 53 is formed as the mounting base. As illustrated in FIG. 7, the side wall of the accommodation groove 53 is fixed to the outer housing 1 through an engagement. Of course, the support member 5 may also be fixed to the outer housing 1 by other means.

While some embodiments of the present disclosure have been described above, the present disclosure is not limited to these embodiments. Various changes and variations of the present disclosure are conceivable for those skilled in the art. Any modifications, equivalent replacements, and improvements made within the spirit and the principle of the present disclosure shall fall within the scope of the claims of the present disclosure.

What is claimed is:

1. A continuous analyte monitoring device, comprising:
an outer housing having a first end and a second end that are arranged opposite to each other in a first direction, the second end having an implantation opening;
a cover detachably connected to the second end and covering the implantation opening;
a driving unit disposed inside the outer housing and movable relative to the outer housing along the first direction;
a puncture unit disposed inside the outer housing, the puncture unit being movable along the first direction with the driving unit and movable relative to the driving unit along a second direction, the second direction being opposite to the first direction; and
a monitoring unit comprising a first monitoring component and a second monitoring component that are spaced apart from each other inside the outer housing along the first direction, the first monitoring component comprising a sensor, and the second monitoring component comprising a signal processing module, wherein
prior to use, remove the cover and perform a single trigger to enable the driving unit to move along the first direction, thereby partially inserting the sensor into a host and establishing an electrical connectivity between the first monitoring component and the second monitoring component.

2. The continuous analyte monitoring device according to claim 1, wherein the first monitoring component further comprises a battery configured to be electrically connected to the second monitoring component.

3. The continuous analyte monitoring device according to claim 1, wherein the first monitoring component comprises an upper housing, the sensor being disposed at the upper housing, the second monitoring component further comprises a lower housing, the signal processing module being disposed at the lower housing, and the upper housing and the lower housing are connected to each other to enable the upper housing and the lower housing to be sealed.

4. The continuous analyte monitoring device according to claim 1, wherein the outer housing comprises a first housing comprising a connection portion, and a second housing connected to the connection portion, wherein the cover is detachably connected to the second housing to cover the implantation opening, the first monitoring component is fixed inside the first housing, and the second monitoring component is fixed inside the second housing.

5. The continuous analyte monitoring device according to claim 4, wherein the second housing comprises a fixing portion configured for interference fit with the second monitoring component, and the cover is equipped with a support post extending towards the outer housing and in contact with the second monitoring component to support the second monitoring component.

6. The continuous analyte monitoring device according to claim 1, further comprising:
a pushing member configured to drive the driving unit to move along the first direction; and
a restoring member configured to drive the puncture unit to move along the second direction, wherein the pushing member and the restoring member at least partially overlap in the first direction.

7. The continuous analyte monitoring device according to claim 1, further comprising a trigger unit disposed at the first end, the trigger unit being movable or deformable along the first direction to trigger the driving unit, to enable the first monitoring component or the second monitoring component to move along the first direction.

8. The continuous analyte monitoring device according to claim 1, wherein the driving unit comprises a first mounting groove internally provided with a restoring member configured to drive the puncture unit to move along the second direction, the puncture unit being disposed in the first mounting groove, and a second mounting groove surrounding an outer periphery of the first mounting groove and internally provided with a pushing member configured to drive the driving unit to move along the first direction.

9. The continuous analyte monitoring device according to claim 8, wherein the puncture unit comprises a puncture needle, and a needle hub having an engagement groove and at least partially overlapping the driving unit in the first direction, the restoring member being disposed in the engagement groove.

10. The continuous analyte monitoring device according to claim 9, further comprising a support member disposed inside the outer housing and comprising a first positioning portion and a second positioning portion, the first positioning portion being engaged with the first mounting groove to form a first guiding channel configured to receive the restoring member, and the second positioning portion being engaged with the second mounting groove to form a second guiding channel configured to receive the pushing member.

11. The continuous analyte monitoring device according to claim 10, wherein the first positioning portion comprises a positioning protrusion protruding towards the engagement groove, and the restoring member has an end connected to the positioning protrusion and another end connected to the puncture unit.

12. The continuous analyte monitoring device according to claim 1, wherein the driving unit is provided with a stop portion configured to abut and engage with the puncture unit and apply a force in the first direction to the puncture unit, and the continuous analyte monitoring device further comprises an unlocking structure located in a movement path of the stop portion and configured to drive the stop portion to disengage from the puncture unit.

13. The continuous analyte monitoring device according to claim 12, wherein the stop portion comprises a fixed end and a movable end configured to swing around the fixed end and provided with a stop segment and a trigger segment, the stop segment extending towards the puncture unit and being configured to abut and engage with the puncture unit, and the trigger segment being configured to engage with the unlocking structure.

14. The continuous analyte monitoring device according to claim 1, further comprising a locking member disposed inside the outer housing, the locking member being elastically deformable or movable relative to the outer housing to have a locked state in which the locking member is engaged with the driving unit to restrict a movement of the driving unit and an unlocked state in which the locking member is disengaged from the driving unit.

15. The continuous analyte monitoring device according to claim 14, wherein the outer housing is internally provided with a mounting base, the locking member comprises a trigger portion located at a side of the mounting base and an acting portion located at another side of the mounting base and engaged with the driving unit, the continuous analyte monitoring device further comprises a trigger unit disposed at the first end and engaged with the trigger portion, and the acting portion is configured to swing around the mounting base to switch into the unlocked state when the trigger portion is pressed by the trigger unit.

16. A continuous analyte monitoring device, comprising:
an outer housing having a first end and a second end that are arranged opposite to each other in a first direction, the second end having an implantation opening;
a driving unit disposed inside the outer housing and movable relative to the outer housing along the first direction;
a puncture unit disposed inside the outer housing, the puncture unit being movable along the first direction with the driving unit and movable relative to the outer housing along a second direction, the second direction being opposite to the first direction; and
a monitoring unit comprising a first monitoring component and a second monitoring component that are spaced apart from each other inside the outer housing, wherein
a single trigger is performed to move the driving unit along the first direction to partially insert a sensor into a host and electrically connect the first monitoring component with the second monitoring component.

17. The continuous analyte monitoring device according to claim 16, wherein the first monitoring component further comprises a battery configured to be electrically connected to the second monitoring component.

18. The continuous analyte monitoring device according to claim 16, wherein—the first monitoring component is configured to move along the first direction to establish an electrical connectivity with the second monitoring component.

19. The continuous analyte monitoring device according to claim 16, further comprising:
a pushing member configured to drive the driving unit to move along the first direction, and
a restoring member configured to drive the puncture unit to move along the second direction, wherein the pushing member and the restoring member at least partially overlap in the first direction.

20. A continuous analyte monitoring device, comprising:
an outer housing having a first end and a second end that are arranged opposite to each other in a first direction, the second end having an implantation opening;
a driving unit disposed inside the outer housing and movable relative to the outer housing along the first direction;
a puncture unit disposed inside the outer housing, the puncture unit being movable along the first direction with the driving unit and movable relative to the outer housing along a second direction, the second direction being opposite to the first direction; and
a monitoring unit comprising a first monitoring component and a second monitoring component that are spaced apart from each other inside the outer housing along the first direction, the first monitoring component comprising a sensor, and the second monitoring component comprising a signal processing module, wherein
a single trigger is performed to move the driving unit along the first direction to partially insert the sensor into a host and enable the first monitoring component to be fixed to the second monitoring component in the first direction.
